(19) United States
(12) Patent Application Publication
(10) Pub. No.: US 2018/0008612 A1
(43) Pub. Date: Jan. 11, 2018

(54) TRANSDERMAL DELIVERY SYSTEM CONTAINING GALANTAMINE OR SALTS THEREOF

(71) Applicant: TAHO Pharmaceuticals Ltd., Taipei City (TW)

(72) Inventors: Catherine Lee, West Linn, OR (US); Taijung Wu, Taoyuan City (TW)

(21) Appl. No.: 15/643,290

(22) Filed: Jul. 6, 2017

Related U.S. Application Data
(60) Provisional application No. 62/360,560, filed on Jul. 11, 2016.

(51) Int. Cl. A61K 31/55; A61K 47/22; A61K 47/20; A61K 47/14; A61K 9/70; A61K 47/12; A61K 47/10; A61K 47/02; A61M 37/00; A61K 47/16

(52) U.S. Cl. CPC A61K 31/55; A61M 37/00; A61K 47/22; A61K 47/20; A61K 47/16; A61K 9/7053; A61K 47/12; A61K 47/10; A61K 47/02; A61K 9/7061; A61K 47/14

(57) ABSTRACT

Disclosed herein is a transdermal delivery system comprising galantamine or its salt as an active ingredient. Also provided are methods of delivering a therapeutically effective amount of galantamine to a subject for the treatment of a disease condition. The disease condition includes a neurological condition such as Alzheimer's disease. Kits including the transdermal delivery system and methods of making such delivery system are also provided.

### 1. INTRODUCTION

Disclosed herein is a transdermal delivery system comprising galantamine or its salt as an active ingredient. Also provided are methods of delivering a therapeutically effective amount of galantamine to a subject for the treatment of a disease condition. The disease condition includes a neurological condition such as Alzheimer's disease. Kits including the transdermal delivery system and methods of making such delivery system are also provided.

### 2. BACKGROUND

It has been reported that Alzheimer's disease is the most common type of neurological disease characterized by complex perception disorder such as losses in psychological ability, memory loss, retrogress of intelligence, changes in personality, and abnormal activity, and it occurs mostly in old age. It has been reported that the average survival period of such patients is known to be around eight years. Brookmeyer R, Corrada M M, Curriero F C, Kawas C. Survival following a diagnosis of Alzheimer disease. Arch Neurol 2002; 59(11):1764-7.

It has been reported that though the pathogenesis of Alzheimer's disease has not been fully investigated, it is known that acetylcholine, which is a neurotransmitter, and the concentration of acetylcholine transferase, which synthesizes acetylcholine (Ach), is reduced by around 16-30% in the brain of dementia patients than in normal persons. Nordberg A and Svensson A L. Cholinesterase inhibitors in the treatment of Alzheimer's disease: a comparison of tolerability and pharmacology. Drug Saf 1998; 19: 465-480. One of the treatments recently being investigated involves inhibiting cholinesterase that is responsible for hydrolysis of acetylcholine. Through inhibiting cholinesterase, neurons are activated by increasing acetylcholine in the brain of patients. Galantamine (brand name: Reminyl®), Donepezil (brand name: Aricept®), and Rivastigmin (brand name: Exelon®) are among cholinesterase inhibitors that are used.

It has been reported that Galantamine, a teriary alkaloid, is an acetyl cholinesterase inhibitor that is marketed as a tablet or liquid formulation for oral administration. It is used to treat mild to moderate symptoms of Alzheimer's disease. Giacobini 2000: “Cholinesterase inhibitors stabilize Alzheimer disease.”. Neurochemical Research vol. 25 (9-10): 1185-1190. When orally administered, galantamine undergoes a first-pass metabolism, which is believed to cause side effects such as abdominal pain, nausea, vomiting, diarrhea and lack of appetite. Reminyl Product Information, Razadyne Product Information.

It has been reported that if Galantamine is administered as a transdermal drug delivery system, not only these side effects related with gastric diseases can be reduced, but also it can provide benefits such as avoiding first pass metabolism, improvement in bioavailability, and offering regulated drug concentration in the plasma. U.S. Pat. No. 5,700,480 discloses a transdermal drug delivery system including a drug reservoir layer comprising Galantamine, plasticizer, and polyacrylate (for example, acrylate copolymer/methacrylate copolymer) as an adhesive. However, this system has transmittance as low as 2.7 μg/cm²/hr. Such low drug transmittance is inefficient in delivering galantamine to the patient for meaningful treatment of the disease.

Therefore, there is a need to develop an optimal galantamine formulation with high efficiency, increased drug loading capacity and drug release sustainability so as to improve the lives of Alzheimer's patients. The formulation disclosed herein helps to eliminate or minimize the side effects associated with oral administration and achieve the desired skin penetration rate and adhesion property. Galantamine and its pharmaceutically acceptable salt easily crystalize and generally have low permeation rate. In addition, galantamine and its pharmaceutically acceptable salt degrade quickly in room temperature and are easily oxidized in formulations. It has been a challenge to provide a stable galantamine transdermal delivery system that does not have crystallization and oxidization problems and yet have an acceptable high drug permeation rate.

### 3. SUMMARY

An object of the present disclosure is to provide a method and a device for transdermal delivery of galantamine or its salt as an active ingredient to a subject through skin or other body surface.

The transdermal delivery system consists of a backing layer, the drug-containing matrix layer, and a release layer. The subject can wear the device over an extended period of time without side effects.

Also disclosed is a transdermal system having improved drug and enhancer loading with desirable tack and adhesion. In one embodiment, the transdermal system comprises one type of copolymer as an adhesive.

Further provided herein is a drug delivery system and method for controlled and sustained delivery of therapeutic amounts of galantamine or its salt to a subject. In certain embodiments, the drug delivery system comprises a matrix which comprises galantamine or its salt dispersed therein. In certain embodiments, the system and method include a matrix formed of a polymer and galantamine or its salt dispersed uniformly within the polymer.

In certain embodiments, provided herein are a transdermal delivery system which comprises a drug-containing matrix layer comprising: (i) galantamine or its pharmaceutically acceptable salt; (ii) an adhesive comprising an acrylic polymer without a functional group or an acrylate-vinyl acetate polymer having at least one carboxyl functional group (COOH); (iii) an enhancer composition including: (a) oleyl oleate and oleic acid; or (b) oleyl oleate and propylene glycol and an antioxidant; or (c) a medium chain fatty acid triglyceride and an antioxidant. In one embodiment, the enhancer composition comprises oleyl oleate and propyleneglycol monolaurate and an antioxidant.

In certain embodiments, the medium chain fatty acid triglyceride comprises about 50-80% of caprylic acid and about 20-50% of capric acid. In certain embodiments, the galantamine or its pharmaceutically acceptable salt is about 7 to about 14% by weight based on the total weight of the drug-containing matrix layer. In certain embodiments, the galantamine or its pharmaceutically acceptable salt is about 7, about 8 or about 14% by weight based on the total weight of the drug-containing matrix layer. In certain embodiments, the adhesive is about 75-78% by weight based on the total weight of the drug-containing matrix layer. In certain embodiments, the acrylic polymer has a glass transition temperature from about −15° C. to about −30° C. In certain embodiments, the acrylic polymer is Duro Tak® 87-9301. In certain embodiments, the acrylate polymer has a glass transition temperature from about −30° C. to about −60° C. In certain embodiments, the acrylate polymer containing vinlyacetate is Duro Tak® 387-2054. In certain embodiments, the medium chain fatty acid triglyceride is Labrafac™ lipophile WL1349. In certain embodiments, the oleic acid is about 10% by weight based on the total weight of the drug-containing matrix layer. In certain embodiments, the oleyl oleate is about 5% by weight based on the total weight of the drug-containing matrix layer. In certain embodiments, the medium chain fatty acid triglyceride is about 10% by weight based on the total weight of the drug-containing matrix layer. In certain embodiments, the medium chain fatty acid triglyceride is Labrafac™ lipophile WL1349. In certain embodiments, the antioxidant is butylated hydoxytoluene. In certain embodiments, the antioxidant is about 0.05% by weight based on the total weight of the drug-containing matrix layer. In certain embodiments, the drug-containing matrix layer has a thickness from about 20 μm to about 48 μm. In certain embodiments, the system has a flux of about 8-10 μg/cm² hr. In certain embodiments, the galantamine or its pharmaceutically acceptable salt is about 7-8% by weight, wherein the adhesive is about 78% by weight, wherein the oleic acid is about 10% by weight, wherein the oleyl oleate is about 5% by weight, based on the total weight of the drug-containing matrix layer, wherein the drug-containing matrix layer has a thickness from about 20-48 μm.

In certain embodiments provided herein are a transdermal delivery system which comprises a drug-containing matrix layer comprising: (i) about 7-8% of galantamine or its pharmaceutically acceptable salt; (ii) an adhesive comprising an acrylic polymer without a functional group; (iii) an enhancer composition comprising: (a) about 10% oleic acid and about 5% oleyl oleate, wherein the transdermal system has a thickness of about 20-48 μm. In certain embodiments, the adhesive has a glass transition temperature from about −15° C. to about −30° C. In certain embodiments, the adhesive is Duro Tak® 87-9301.

In certain embodiments provided herein are a transdermal delivery system which comprises a drug-containing matrix layer comprising: (i) about 14% of galantamine or its pharmaceutically acceptable salt; (ii) an adhesive comprising an acrylate-vinlyacetate polymer having at least one functional COOH group; (iii) an enhancer composition comprising about 10% medium chain fatty acid triglyceride: (iv) about 0.05% antioxidant, wherein the transdermal system has a thickness of about 15-45 μm. In certain embodiments, the adhesive has a glass transition temperature from about −30° C. to about −60° C. In certain embodiments, the adhesive is Duro Tak® 387-2054. In certain embodiments, the medium chain fatty acid triglyceride is Labrafac™ lipophile WL1349. In certain embodiments, the antioxidant is butylated hydroxytoluene, vitamin E, ascorbyl palmitate, bronopol, erythorbic acid, monothioglycerol, sodium metabisulfite, sodium sulfite, sulfur dioxide, sodium thiosulfate or combinations thereof. In certain embodiments, the system has a flux of about 8 μg/cm² hr.

In certain embodiments provided herein are a transdermal delivery system which comprises a drug-containing matrix layer comprising: (i) about 14% of galantamine or its pharmaceutically acceptable salt; (ii) an adhesive comprising an acrylate-vinlyacetate polymer having at least one functional COOH group; (iii) an enhancer composition comprising about 5% polypylene glycol and about 5% oleyl oleate; (iv) about 0.05% antioxidant, wherein the transdermal system has a thickness of about 15-45 μm. In certain embodiments, the adhesive has a glass transition temperature from about −30° C. to about −60° C. In certain embodiments, the adhesive is Duro Tak® 387-2054. In certain embodiments, the system has a flux of about 9.3 to about 9.5 μg/cm² hr.

Provided herein is a transdermal delivery system comprising a drug-containing matrix layer comprising: (a) galantamine or its pharmaceutically acceptable salt that is about 14% by weight; (b) adhesive that is about 76% by weight; (c) oleyl oleate that is about 5% by weight; (d) medium chain fatty acid triglyceride that is about 10%; (e) polyethylene glycol that is about 5%; (f) an antioxidant including butylated hydroxytoluene that is about 0.05% by weight, based on the total weight of the drug-containing matrix layer. In certain embodiments, the drug-containing matrix layer has a thickness of about 15-45 μm. In certain embodiments, the system has a flux of about 1-15 μg/cm² hr. In certain embodiments, the medium chain fatty acid triglyceride is Labrafac™ lipophile WL1349.

In certain embodiments provided herein are a transdermal delivery system which comprises a drug-containing matrix layer comprising: (i) galantamine or its pharmaceutically acceptable salt; (ii) an adhesive comprising styrene butadiene-styrene block copolymer (“SBS block copolymer”); (iii) an enhancer composition comprising oleyl oleate and propyleneglycol monolaurate; and (iv) an antioxidant, a solvent and an adhesive modifier. In certain embodiments, the antioxidant is butylated hydroxytoluene, the solvent is diethylene glycol monoethyl ether and the adhesive modifier is a cyclic terpene. In certain embodiments, the antioxidant is vitamin E, ascorbyl palmitate, bronopol butylated hydroxytoluene (BHT), erythorbic acid, monothioglycerol, sodium metabisulfite, sodium sulfite, sulfur dioxide, sodium thiosulfate and combinations thereof. In certain embodiments, the galantamine or its pharmaceutically acceptable salt is about 0.01 to about 5% by weight based on the total weight of the drug-containing matrix layer. In certain embodiments, the styrene-butadiene-styrene block copolymer is about 60-97.5% by weight based on the total weight of the drug-containing matrix layer. In certain embodiments, the enhancer composition is about 3-15% by weight based on the total weight of the drug-containing matrix layer. In certain embodiments, the propyleneglycol monolaurate is about 0.1-15% by weight based on the total weight of the drug-containing matrix layer. In certain embodiments, the oleyl oleate is about 0.1-20% by weight based on the total weight of the drug-containing matrix layer. In certain embodiments, the antioxidant is butylated hydroxytoluene and is about 0.001-0.5% by weight based on the total weight of the drug-containing matrix layer. In certain embodiments, the solvent is diethylene glycol monoethyl ether and is about 0.1-20% by weight based on the total weight of the drug-containing matrix layer. In certain embodiments, the adhesive modifier is a cyclic terpene and is about 0.1-15% by weight based on the total weight of the drug-containing matrix layer. In certain embodiments, the propyleneglycol monolaurate is propyleneglycol monolaurate type I or propyleneglycol monolaurate type II. In certain embodiment, the drug-containing matrix layer has a thickness from about 45 μm to about 85 μm. In certain embodiments, the galantamine or its pharmaceutically acceptable salt is about 2.5% by weight, wherein the styrene-butadiene-styrene block copolymer is about 60-97.5% by weight, wherein the propyleneglycol monolaurate is about 0.1-15% by weight, wherein oleyl oleate is about 0.1-20% by weight, wherein the antioxidant is butylated hydroxyl toluene and is about 0.001-0.5% by weight, wherein the solvent is diethylene glycol monoethyl ether and is about 0.1-20% by weight, and wherein the adhesive modifier is cyclic terpene and is about 0.1-15% by weight, based on the total weight of the drug-containing matrix layer. In certain embodiments, the propyleneglycol monolaurate is propyleneglycol monolaurate type I or propyleneglycol monolaurate type II.

In certain embodiments provided herein are a transdermal delivery system which comprises a drug-containing matrix layer comprising: (i) about 0.01-5% of galantamine or its pharmaceutically acceptable salt; (ii) about 60-97.7% of an adhesive comprising styrene butadiene-styrene block copolymer, (iii) an enhancer composition comprising about 0.1-20% oleyl oleate and about 0.1-15% propyleneglycol monolaurate; (v) about 0.001-0.5% of an antioxidant; (vi) about 0.1-20% solvent: and (vii) about 0.1-15% adhesive modifier.

In certain embodiments, the galantamine is about 2.5% by weight based on the total weight of the drug-containing matrix layer. In certain embodiments, the enhancer composition is about 3-8% by weight based on the total weight of the drug-containing matrix layer. In certain embodiments, the propyleneglycol monolaurate is propyleneglycol monolaurate type I or propyleneglycol monolaurate type II. In certain embodiments, the antioxidant is butylated hydroxytoluene, vitamin E, ascorbyl palmitate, bronopol, erythorbic acid, monothioglycerol, sodium metabisulfite, sodium sulfite, sulfur dioxide, sodium thiosulfate and combinations thereof. In certain embodiments, the solvent is diethylene glycol monoethyl ether. In certain embodiments, the adhesive modifier is cyclic terpene. In certain embodiments, the enhancer composition is about 3, about 5 or about 8% by weight based on the total weight of the drug-containing matrix layer.

Also provided herein is a method of making the transdermal delivery system. In some embodiments, the method includes a controlled and sustained drug delivery system useful to deliver Galantamine and its salt to a patient. The system comprises delivery of substantially homogeneous particles. In certain embodiments, the substantially homogeneous particles are dispersed in a polymeric matrix. Also disclosed is the method of preparing a polymeric matrix.

When compared with other galantamine transdermal delivery system, the present disclosure provides 2-3, 3-4, 4-5, 5-6, 6-7, 7-8, 8-9, 9-10, 10-11, or 11-12 folds higher release rate as compared with other transdermal delivery system.

Provided herein is a method for delivering drugs to a patient comprising the step of administering the transdermal delivery system disclosed herein.

Also described herein is a transdermal delivery system used to treat or prevent diseases in a subject. In a specific embodiment, the subject is a mammal. In a specific embodiment, the subject is human. In one embodiment, provided herein is a method of treating a disease comprising administering to a subject, a transdermal delivery system comprising a therapeutically effective amount of galantamine or its salt. Provided herein is a method of making the transdermal delivery system.

Also disclosed is a kit comprising the transdermal delivery system provided herein. The kit comprises a carrier being compartmentalized to receive in close confinement one or more containers comprising one of the separate elements to be used in the method. The kit also contains instructions for administering the transdermal delivery system.

A kit as provided herein comprises a unit dose of galantamine or its salt provided herein, such that when administered to a subject, a therapeutically or prophylactically effective plasma level of the compound or composition can be maintained in the subject for at least 1, 3, 5, and 7 day.

These and other aspects, features, and advantages can be appreciated from the accompanying description of certain embodiments of the disclosure and the accompanying drawing figures and claims.

### 4.1 DEFINITIONS

As used herein, the terms “subject” and “patient” are used interchangeably. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In one embodiment, the subject is a human.

“Treating” or “treatment” of any disease or disorder refers, in one embodiment, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

As used herein, the terms “preventing” and “prevention” of any disease and disorder refers to the prevention of a disorder or one or more symptoms thereof. Preventing and prevention is to impede the onset, development, and progression of disorder or symptoms.

As used herein, the term “about” is defined as a deviation of +5% from a numerical value.

As used herein, the term “acrylate-vinyl acetate polymer” is defined as copolymer of acrylic soft and hard monomers containing vinyle acetate polyacrylates.

As used herein, the term “enhancer composition” is defined as a composition comprising one or more enhancers for improving penetration of an active agent.

As used herein, the term “medium chain fatty acid triglyceride is defined as triglycerides whose fatty acids have an aliphatic tail of 6-12 carbon atoms. The medium chain fatty acid triglyceride consists mainly of mixture of triglycerides of saturated fatty acids including caprylic acid in the range of 50-80% and capric acid in the range of 20-50%. In certain embodiments, the medium chain fatty acid triglyceride is Labrafac™ lipophile WL1349.

### 4.2 DETAILED DESCRIPTION OF THE DRAWINGS

FIG. 1 shows various amount of galantamine in acrylate-vinyl acetate copolymer.

FIG. 2 shows various amount of galantamine in styrene-butadiene block copolymer rubber.

### 5. DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill have not been described in detail so as not to obscure claimed subject matter. It is to be understood that particular features, structures, or characteristics described may be combined in various ways in one or more implementations.

### 5.1 Components of the Transdermal Delivery System

The transdermal delivery system described herein comprises a drug-containing matrix layer, a backing layer and a release layer. The drug-containing matrix layer comprises galantamine or its pharmaceutically acceptable salt. A “pharmaceutically acceptable salt” means a galantamine salt that induces a desired pharmacological or physiological effect, and include agents that are therapeutically effective or prophylactically effective. In certain embodiments, the drug-containing matrix layer includes one or more active agents, which agents are galantamine and its pharmaceutically acceptable salt.

### 5.1.1 Drug-Containing Matrix Layer

The amount of galantamine and its pharmaceutically acceptable salt present in the drug-containing matrix layer may vary. In certain embodiments, the amount of galantamine and its pharmaceutically acceptable salt deliver galantamine ranges from about 4 mg to about 24 mg. In certain embodiments, the galantamine and its pharmaceutically acceptable salt ranges from about 4-8 mg, about 8-10 mg, about 10-12 mg, about 12-14 mg, about 14-16 mg, about 16-18 mg, about 18-20 mg, or about 20-24 mg per unit of the transdermal delivery system. In one embodiment, a unit of the transdermal delivery system is one dose of the transdermal delivery system. In one embodiment, one dose is one patch. In certain embodiments, the galantamine and its pharmaceutically acceptable salt present in the drug-containing matrix layer is about 0.01-0.05%, about 0.05%-0.1%, about 0.1-0.2%, about 0.2-0.5%, about 0.5-1%, about 1-2%, about 2-4%, about 2-5%, about 5-6%, about 6-7%, about 7-8%, about 8-9%, about 9-10%, about 10-13%, about 13-14%, about 14-15%, about 15-16%, about 16-17%, about 17-18%, about 18-19%, about 19-20%, or about 20-25%, by weight based on the total weight of the drug-containing matrix layer. In certain embodiments, the galantamine and its pharmaceutically acceptable salt is about 0.01-5% by weight based on the total weight of the drug-containing matrix layer. In certain embodiments, the galantamine and its pharmaceutically acceptable salt is about 1-16% by weight based on the total weight of the drug-containing matrix layer. In certain embodiments, the galantamine and its pharmaceutically acceptable salt is about 0.5-1, 1-2.5, 2.5-3, 3-4, 4-5, 5-6, 6-7, 7-8, 8-9, 9-10, 10-11, 11-12, 12-13, or 13-14% by weight based on the total weight of the drug-containing matrix layer.

In certain embodiments, the drug-containing matrix layer comprises an adhesive. In certain embodiments, the adhesive present in the drug-containing matrix layer is about 10-30%, about 30-50%, about 50-60%, about 60-65%, about 65-70%, about 70-75%, about 75-80%, about 75-80%, about 80-90%, about 90-95%, about 95-96%, about 96-97%, about 97-98%, about 98-99%, by weight based on the total weight of the drug-containing matrix layer. In certain embodiments, the adhesive is about 75-85% by weight based on the total weight of the drug-containing matrix layer. In certain embodiments, the adhesive is about 60-97.5% by weight based on the total weight of the drug-containing matrix layer.

In certain embodiments, the adhesive present in the drug-containing matrix layer comprises a polymer. In certain embodiments, the polymer is an acrylic polymer, or an acrylate-vinyl acetate polymer. In certain embodiments, the adhesive is an acrylic polymer without a functional group. In certain embodiments, the adhesive is an acrylic polymer with one or more functional groups including —COOH or —OH. In certain embodiments, the adhesive is an acrylate-vinyl acetate polymer with one or more functional groups including —COOH or —OH. In certain embodiments, the acrylic polymer without a functional group has a gas transition temperature from about −15° C. to about −30° C. In certain embodiments, the acrylic polymer with a functional group has a gas transition temperature from about −30° C. to −60° C. In certain embodiments, the acrylic polymer with a functional group has a gas transition temperature from about −30° C. to −60° C. In certain embodiments, the acrylate-vinyl acetate polymer with a functional group has a gas transition temperature from about −15° C. to about −30° C. In certain embodiments, the acrylate-vinyl acetate polymer with a functional group has a gas transition temperature from about −30° C. to −60° C.

In certain embodiments, the adhesive comprises styrene butadiene-styrene block copolymer.

In certain embodiments, the polymers include, but are not limited to, polyurethanes, acrylates, styrenic block copolymers and silicones. In certain embodiments, the polymer includes, but are not limited to, an acrylate polymer, polysiloxanes, polyisobutylene (PIB), polyisoprene, polybutadiene, styrenic block polymers and combinations thereof. Suitable styrenic block copolymer-based adhesives include, but are not limited to, styrene-isoprene-styrene block copolymer (SIS), styrene-butadiene-styrene copolymer (SBS), styrene-ethylenebutene-styrene copolymers (SEBS), and di-block analogs and combinations thereof.

The drug-containing matrix layer may include a pressure sensitive adhesive. The terms “pressure sensitive adhesive” means an adhesive that forms an adhesive bond when pressure is applied to adhere the adhesive with a surface. In certain embodiments, the degree of bond strength is proportional to the amount of pressure that is used to apply the adhesive to the surface. Pressure sensitive adhesives include, but are not limited to, acrylate polymers.

Acrylate polymers may include copolymers of various monomers which may be “soft” monomers or “hard” monomers or combinations thereof. Soft monomers are characterized by having a relatively lower glass transition temperature, and include examples such as, but not limited to, n-butyl acrylate, 2-ethylhexyl acrylate and isooctyl acrylate. Hard monomers are characterized by having a relatively higher glass transition temperature, and include examples, such as, but not limited to include styrene, methyl methacrylate, ethyl acrylate and methyl acrylate. The acrylate polymers can be composed of a copolymer including bipolymer (e.g., made with two monomers), a terpolymer (e.g., made with three monomers), or a tetrapolymer (e.g., made with four monomers), or copolymers made from greater numbers of monomers. The acrylate polymers can include cross-linked and non-cross-linked polymers. The polymers can be cross-linked by cross-linking agents to provide the desired cross-linked polymers. In certain embodiments, the polymers are not cross-linked. In certain embodiments, the polymers are cured. In certain embodiments, the polymers are not cured.

Monomers from which the acrylate polymers are produced include at least two or more components selected from acrylic acids, alkyl acrylates, methacrylates. Additional examples of acrylic adhesive monomers are described in Satas, “AcrylicAdhesives,” Handbook of Pressure-Sensitive Adhesive Technology, 2nd ed., pp. 396-456 (D. Satas, ed.), Van Nostrand Reinhold, New York (1989).

In certain embodiments, the adhesive comprises Duro Tak® 87-9301 (Henkel), Duro Tak® 387-2054 (Henkel) or styrene butadiene-styrene block copolymer.

In certain embodiments, the adhesive comprises Duro Tak® 87-4098, Duro Tak® 87-2510, Duro Tak® 87-2516, Duro Tak® 87-4287, Duro Tak® 87-235A, Duro Tak® 87-2852. Duro Tak® 87-2353, Duro Tak® 87-2196, Duro Tak® 87-2979, Duro Tak® 87-502b, Duro Tak® 87-504b, Duro Tak® 87-6908, Duro Tak® 87-6911, BIO-PSA®4102 and BIO-PSA®4602.

In certain embodiments, the adhesive that are useful includes those disclosed in Tables 3, 5-11, 12 and 16 infra.

In certain embodiments, the drug-containing matrix layer comprises an enhancer composition that enhances drug permeation. The enhancer composition facilitates the absorption of the active agent through the skin of the subject.

In certain embodiments, the enhancer composition comprises: (a) oleyl oleate and oleic acid; (b) oleyl oleate and propylene glycol and an antioxidant; or (c) a medium chain fatty acid triglyceride and an antioxidant. In certain embodiment, the medium chain fatty acid triglyceride consists mainly of mainly consist of mixture of triglycerides of saturated fatty acids including caprylic acid in the range of 50-80% and capric acid in the range of 20-50%. In certain embodiments, the medium chain fatty acid triglyceride is Labrafac™ lipophile WL1349.

In certain embodiments, the enhancer composition comprises oleyl oleate and propyleneglycol monolaurate. In certain embodiments, the propyleneglycol monolaurate is type I or Type II. In certain embodiments, the propyleneglycol monolaurate is Lauroglycol™ FCC or Lauroglycol™ 90. In certain embodiments, the propylene glycol monolaurate is a mixture of the propylene glycol mono and di-esters of lauric acid. In certain embodiments, the enhancer composition comprises type I propyleneglycol monolaurate which has a range of mono-ester content 45-70%, and di-ester content of 30-55%. In certain embodiments, the enhancer composition comprises type II propylene glycol monolaurate which has mono-ester content of 90-100% and di-ester content of 0-10%.

In certain embodiments, suitable enhancer compositions may include, but is not limited to, aliphatic alcohols, such as but not limited to saturated or unsaturated higher alcohols having 12 to 22 carbon atoms, such as oleyl alcohol and lauryl alcohol; fatty acids, such as but not limited to linolic acid, oleic acid, linolenic acid, stearic acid, isostearic acid and palmitic acid; fatty acid esters, such as but not limited to isopropyl myristate, diisopropyl adipate, and isopropyl palmitate; alcohol amines, such as but not limited to triethanolamine, triethanolamine hydrochloride, and diisopropanolamine; polyhydric alcohol alkyl ethers, such as but not limited to alkyl ethers of polyhydric alcohols such as glycerol, ethylene glycol, propylene glycol, 1,3-butylene glycol, diglycerol, polyglycerol, diethylene glycol, polyethylene glycol, dipropylene glycol, polypropylene glycol, sorbitan, sorbitol, isosorbide, methyl glucoside, oligosaccharides, and reducing oligosaccharides, where the number of carbon atoms of the alkyl group moiety in the polyhydric alcohol alkyl ethers is preferably 6 to 20; polyoxyethylene alkyl ethers, such as but not limited to polyoxyethylene alkyl ethers in which the number of carbon atoms of the alkyl group moiety is 6 to 20, and the number of repeating units (e.g. —OCH2CH2-) of the polyoxyethylene chain is 1 to 9, such as but not limited to polyoxyethylene lauryl ether, polyoxyethylene cetyl ether, polyoxyethylene stearyl ether, and polyoxyethylene oleyl ether; glycerides (i.e., fatty acid esters of glycerol), such as but not limited to glycerol esters of fatty acids having 6 to 18 carbon atoms, where the glycerides may be monoglycerides (i.e., a glycerol molecule covalently bonded to one fatty acid chain through an ester linkage), diglycerides (i.e., a glycerol molecule covalently bonded to two fatty acid chains through ester linkages), triglycerides (i.e., a glycerol molecule covalently bonded to three fatty acid chains through ester linkages), or combinations thereof, where the fatty acid components forming the glycerides include, but are not limited to octanoic acid, decanoic acid, dodecanoic acid, tetradecanoic acid, hexadecanoic acid, octadecanoic acid (i.e., stearic acid) and oleic acid; middle-chain fatty acid esters of polyhydric alcohols; lactic acid alkyl esters; dibasic acid alkyl esters; acylated amino acids; pyrrolidone; pyrrolidone derivatives: and combinations thereof.

In certain embodiments, suitable enhancer compositions include, but are not limited to lactic acid, tartaric acid, 1,2,6-hexanetriol, benzyl alcohol, lanoline, potassium hydroxide (KOH), and tris(hydroxymethyl)aminomethane. Specific examples of permeation enhancers include, but are not limited to glycerol monooleate (GMO) and sorbitan monolaurate (SML), lactate esters such as lauryl lactate, methyl laurate, caproyl lactic acid, lauramide diethanolamine (LDEA), dimethyl lauramide, polyethylene glycol-4 lauryl ether (Laureth-4), lauryl pyroglutamate (LP), sorbitan monolaurate, ethanol and combinations thereof. In certain embodiments, enhancer compositions enhance permeation of surfactant type active agents. Examples of such enhancer compositions include combinations of semi-polar solvents, e.g., propylene glycol, butane diol, N-methylpyrrolidone, dimethyl sulfoxide, diethylene glycol methyl ether, and dimethyl isosorbide, surfactants, such as isopropyl myristate, oleic acid, lauryl lactate and combinations thereof.

In certain embodiments, enhancer compositions comprises squalane, isopropyl palmitate, isopropyl myristate, sorbitan laurate, DL-limonene, ethyl oleate, methyl dodecanoate, propylene glycol dicaprylocaprate, propylene glycol dicaprylate/dicaprate, Labrafac™ PG, octyl alcohol, dodecyl alcohol, polyoxyethylene (4) lauryl ether, Brij® 30, oleyl alcohol, polyoxyethylene sorbitan monooleate, Tween®80, propylene glycol, diethylene glycol, monoethyl ether, propylene glycol monocaprylate, Capryol PGMC, 1-methyl-2-pyrrolidinone, glyceryl triacetate, triacetin, polyoxyl castor oil, Kolliphor®RH40, oleoyl macrogol-6 glycerides, Labrafil™ M1944CS, linoleoyl polyoxyl-6 glycerides, Labrafil™ M2125CS, caprylocaproyl macrogol-8 glycerides, Labrasol®, polyoxyl castor oil, oleoyl macrogol-6 glycerides, linoleoyl polyoxyl-6 glycerides, caprylocaproyl macrogol-8 glycerides and N-methyl pyrrolidone.

In certain embodiments, the enhancer composition is about 1-3%, about 3-5%, about 5-10%, about 10-15%, about 15-25% by weight based on the total weight of the drug-containing matrix layer. In certain embodiments, the enhancer composition is about 3%, about 5%, about 8%, about 10% by weight based on the total weight of the drug-containing matrix layer.

In certain embodiments, the enhancer useful for the transdermal delivery system includes those disclosed in Table 4 infra.

In certain embodiments, the drug-containing matrix layer comprises one or more antioxidants, such as but not limited to, tocopherol and derivatives, e.g., tocopherol acetate or tocopherol polyethylene glycol succinate, ascorbic acid and derivatives, e.g., ascorbyl palmitate, butylated hydroxyanisole, butylated hydroxytoluene, fumaric acid, malic acid, propyl gallate, metabisulfates and derivatives, etc. In one embodiment, the antioxidant is butylated hydroxytoluene. In certain embodiments, the antioxidant is vitamin E, ascorbyl palmitate, bronopol, erythorbic acid, monothioglycerol, sodium metabisulfite, sodium sulfite, sulfur dioxide, sodium thiosulfate and combinations thereof.

In certain embodiments, the antioxidant may be present in an amount ranging from about 0.001 to 5.0%, about 0.05%, about 0.001-0.005%, about 0.005-0.01%, about 0.01-0.05%, about 0.05-0.1%, about 0.1-0.5%, about 0.5-1%, about 1-3%, about 3-5% by weight based on the total weight of the drug-containing matrix layer.

In certain embodiments, the drug-containing matrix layer may contain fillers which include, but are not limited to: metal oxides (such as zinc oxide and titanium oxide), metal salts (such as calcium carbonate, magnesium carbonate and zinc stearate), silicic acid compounds (such as kaolin, talc, bentonite. Aerosil, hydrous silica, aluminum silicate, magnesium silicate and magnesium aluminometasilicate) and metal hydroxides (such as aluminum hydroxide). Where present, such fillers may be 1 to 75%, such as 2 to 50% by weight based on the total weight of the drug-containing matrix layer.

In certain embodiments, the drug-containing matrix layer includes a solvent which includes but is not limited to a volatile solvent or a non-volatile solvent (i.e., a solvent that is non-volatile as compared to acetone, isopropanol or water, but may nonetheless exhibit some volatility), such as dimethyl sulfoxide (DMSO), N-methylpyrrolidone, dimethyl isosorbide, propylene glycol, hexylene glycol and benzyl alcohol. In one embodiment, the solvent is diethylene glycol monoethyl ether. In certain embodiments, the solvent is Transcutal®P. The drug-containing matrix layer comprises a solvent in an amount of about 0.1-1%, about 1-5%, about 5-10%, about 10-15%, about 15-20%, about 20-30% by weight based on the total weight of the drug-containing matrix layer.

In certain embodiments, the solvents used include, but are not limited to, ethyl acetate, hexane, ethanol, heptane, methanol, cyclohexane, acetyl acetone, xylene, butanone, toluene, 2,4-pentanedione, or isopropyl alcohol.

In certain embodiments, the drug-containing matrix layer includes an adhesion modifier. In one embodiment, the adhesion modifier can be a cyclic terpene or a hydrogenated rosin. In one embodiment, the adhesion modifier is Foral® 85. In one embodiment, the adhesion modifier is Limonene. The drug-containing matrix layer comprises an adhesion modifier in an amount of about 0.1-1%, about 1-5%, about 5-10%, about 10-15%, and about 15-20% by weight based on the total weight of the drug-containing matrix layer.

### 5.2 Delivery of Active Agents

While the actual flux may vary, in certain embodiments, (e.g., as determined using the skin permeation assay in the Section 6 Examples below) skin permeation rates are about 3-4 g/cm²/hr, about 4-5 μg/cm²/hr, about 5-6 μg/cm²/hr, about 6-7 μg/cm²/hr, about 7-8 μg/cm²/hr, about 8-9 μg/cm²/hr, about 9-10 μg/cm²/hr, about 10-11 μg/cm²/hr, or about 11-12 μg/cm²/hr, about 12-15 μg/cm²/hr. about 15-20 μg/cm²/hr, about 20-25 μg/cm²/hr. about 25-30 μg/cm²/hr, about 30-35 μg/cm²/hr. about 35-40 μg/cm²/hr, about 40-45 μg/cm²/hr, or about 45-50 μg/cm²/hr.

In certain embodiments, the flux is about 8 mg/24 hrs/23 cm² to about 24 mg/24 hr/127 cm².

In certain embodiments, the transdermal delivery systems are formulated to provide a cumulative delivered amount (also referred to herein as cumulative flux) of the active agent to a subject when the formulation is applied to the skin of a subject for an extended period of time as described infra. In certain embodiments, the transdermal formulations are configured to provide a cumulative delivered amount of the active agent of about 1-100 μg/cm², about 100-150 μg/cm², about 150-200 μg/cm², about 200-250 μg/cm², about 250-300 μg/cm², about 300-350 μg/cm², about 350-400 μg/cm², about 400-450 μg/cm², about 450-500 μg/cm², about 500-550 μg/cm², about 550-600 μg/cm², about 600-650 μg/cm², 650-700 μg/cm², about 700-750 μg/cm², 750 m-800 μg/cm², 800-850 μg/cm2, about 850-900 μg/cm², about 900-950 μg/cm², or about 950-1000 μg/cm².

The size (i.e., area) of the transdermal delivery system may vary, but is within a range of the active agent to the subject. It is also important that the subject wearing the transdermal delivery system finds the system to be easy to apply and comfortable to use for a period of time so as to improve compliance. In certain embodiments, the size of the formulation is chosen in view of the desired transdermal flux rate of the active agent and the target dosage. In certain embodiments, the transdermal delivery system has a size that is about 2-6 cm², about 6-10 cm², about 10-20 cm², about 20-30 cm², about 30-40 cm², about 40-50 cm², about 50-100 cm², about 100-130 cm², about 130-140 cm², about 140-150 cm² or about 150-200 cm².

The transdermal delivery system of the present disclosure is formulated to provide a therapeutically effective amount of the active agent to a subject when the topical patch is applied to a skin site of a subject for an extended period of time (e.g., a multi-day period of time). For example, the extended period of time may be a period of time that is about 6-12 hours, about 12-24 hours, about 1-2 days, about 2-3 days, about 3-4 days, about 4-5 days, about 5-6 days, about 6-7 days.

In certain embodiments, various formulations, human pharmacokinetics profile and skin permeation properties as shown in Tables 1, 2, 13-14 infra.

The drug-containing matrix layer of the transdermal delivery system may vary in thickness. In certain embodiments, the drug-containing matrix layer has a thickness within a range that is sufficient to provide for the desired extended delivery of a therapeutically effective amount of the active agent to the subject. In certain embodiments, the thickness of the formulation is chosen in view of the desired transdermal delivery rate of the active agent and the target dosage. In certain embodiments, the thickness of the drug-containing matrix layer is about 10 μm to about 15 μm, about 15 μm to about 20 μm, about 20 μm to about 25 μm, about 25 μm to about 30 μm, about 30 μm to about 35 μm, about 35 μm to about 4) μm, about 40 μm to about 45 μm, about 45 μm to about 50 μm, about 50 μm to about 55 μm or about 55 μm to about 120 μm.

An aspect of the transdermal delivery system of the present disclosure is that it may be stored for extended periods of time without significant degradation and/or significant reduction in activity of the active agent. In certain embodiments, the drug-containing matrix layer comprising galantamine or its pharmaceutically acceptable salt is stable for at least 1 year, 2 years, 3 years, 4 years, 5 years, or 6 years. In certain embodiments, the transdermal delivery system is maintained at 25° C.±2° C./60% RH±5% RH.

### 5.3 Multi-Layer Structure of the Transdermal Delivery Device

### 5.3.1. Backing Layer

In certain embodiments, the transdermal delivery system includes a backing layer (e.g., support layer). The backing may be flexible to an extent that it can be brought into close contact with a desired topical location of a subject. The backing may be fabricated from a material that does not absorb the active agent, and does not allow the active agent to be released from the backing side of the transdermal formulation. Backing materials of interest may be occlusive (i.e., impermeable), semi-occlusive or breathable (permeable). The backing may include, but is not limited to, non-woven fabrics, woven fabrics, films (including sheets), foils, porous bodies, foamed bodies, paper, composite materials obtained by laminating a film on a non-woven fabric or fabric, and combinations thereof. Non-woven fabric may include, but is not limited to, the following: polyolefin resins such as polyethylene and polypropylene; polyester resins such as polyethylene terephthalate, polybutylene terephthalate and polyethylene naphthalate; rayon, polyamide, poly(ester ether), polyurethane, polyacrylic resins, polyvinyl alcohol, styrene-isoprene-styrene copolymers, and styrene-ethylene-propylene-styrene copolymers; and combinations thereof.

Fabrics may include, but are not limited to: cotton, rayon, polyacrylic resins, polyester resins, polyvinyl alcohol, and combinations thereof. Films may include, but are not limited to the following: polyolefin resins such as polyethylene (including low density and high density polyethylene (LDPE, HDPE) and polypropylene; polyacrylic resins such as polymethyl methacrylate and polyethyl methacrylate; polyester resins such as polyethylene terephthalate, poly-chloro-tri-fluoro-ethylene, acrylonitrile methyl acrylate copolymer, polybutylene terephthalate and polyethylene naphthalate; and polyvinyl alcohol, ethylene-vinyl alcohol copolymers, polyvinyl chloride, polystyrene, polyurethane, polyacrylonitrile, fluororesins, styrene-isoprene-styrene copolymers, styrene-butadiene rubber, polybutadiene, ethylene-vinyl acetate copolymers, polyamide, and polysulfone; and combinations thereof. Foils may include metallic foils, e.g., aluminum foils, etc. Papers may include, but are not limited to, impregnated paper, coated paper, wood free paper. Kraft paper, Japanese paper, glassine paper, synthetic paper, and combinations thereof. Composite materials may include, but are not limited to, composite materials obtained by laminating the above-described film on the above-described non-woven fabric or fabric. In certain embodiments, the backing includes a polyester, such as polyethylene terephthalate (PET).

In certain embodiments, the backing layer is in contact with a surface of the drug-containing matrix layer. For example, where the transdermal delivery system is configured so that one surface of the drug-containing matrix layer contacts the skin upon application, the backing will be in contact with an opposing surface of the drug-containing matrix layer.

### 5.3.2 Release Layer

In certain embodiments, the transdermal delivery system comprises a release layer. In certain embodiments, a release layer is provided on the drug-containing matrix layer, and specifically on a surface of the drug-containing matrix layer that is distal (i.e., opposite) from the backing layer. The release liner may facilitate the protection of the drug-containing matrix layer before use of the transdermal delivery system. In certain embodiments, the release layer is configured to be removable from the drug-containing matrix layer without retaining the drug-containing matrix layer.

The release layer may be any convenient material. In certain embodiments, the release layer includes polyesters, such as polyethylene terephthalate, polypropylene and combinations thereof. In certain embodiments, the release layer includes a coated substrate, which, for example, may be prepared by treating one side of polyethylene-coated wood free paper, polyolefin-coated glassine paper, a polyethylene terephthalate (polyester) film, a polypropylene film, with a silicone treatment. In certain instances, the release layer includes a polyester film with a silicone treatment.

### 5.4 Methods of Making the Transdermal Delivery System

Aspects of the present disclosure also include methods of producing the transdermal delivery system as disclosed above. In certain embodiments, the method includes mixing galantamine or its pharmaceutically acceptable salt with one or more solvents, an adhesive, an enhancer and an antioxidant to form a drug-containing matrix layer.

In certain embodiments, the adhesive comprises an acrylic polymer without a functional group or an acrylate-vinyl acetate polymer having at least one carboxyl functional group. In certain embodiments, the enhancer composition comprises oleyl oleate and oleic acid. In one embodiment, the enhancer composition comprises oleyl oleate, propylene glycol and an antioxidant. In one embodiment, the enhancer composition comprises a medium chain fatty acid triglyceride and an antioxidant. In certain embodiments, the medium chain fatty acid triglyceride is Labrafac™ lipophile WL1349. In one embodiment, the enhancer composition comprises oleyl oleate and propyleneglycol monolaurate and an antioxidant.

The mixture is then applied to a backing. The method may further include applying a release liner to the drug-containing matrix layer on the side that is opposite to the backing. In certain instances, the method of making the transdermal delivery system further includes placing the transdermal formulation into a package forming a kit. After placing the transdermal formulation into the package, the method may include sealing the package.

### 5.5 Dose

The amount of a composition that will be effective in the treatment or prophylactic treatment of Alzheimer's disease can be determined by standard clinical techniques. In addition, in vitro or in vivo assays may optionally be employed to help identify optimal dosage ranges. The precise dose to be employed will also depend on the seriousness of the disease or disorder, and should be decided according to the judgment of the practitioner and each patient's circumstances.

Suitable doses of the drug for transdermal delivery are in the range of 0.001 milligram to 1 milligram, depending on the area to which the compound is administered. Effective doses may be extrapolated from dose-response curves derived from in vitro or animal model test systems. Such animal models and systems are well known in the art.

### 6. EXAMPLES

The following examples illustrate the synthesis and use of representative embodiments provided herein. These examples are not intended, nor are they to be construed, as limiting the scope of the claimed subject matter. It will be clear that the scope of subject matter may be practiced otherwise than as particularly described herein. Numerous modifications and variations of the subject matter are possible in view of the teachings herein and, therefore, are within the scope the claimed subject matter.

### 6.1 Materials and Methods

The transdermal system are manufactured according to known methodology such as blending (mixing) the polymer(s), drug, and other excipients with appropriate amount in the presence of an appropriate solvent, such as a volatile organic solvent, casting the wet blend onto a release liner, followed by evaporation of the volatile solvent(s) at appropriate drying conditions, laminating the dried drug-in-containing matrix on the release liner onto a backing film.

The flux can be measured with a standard procedure using Franz diffusion cells, and the experiments were done on human cadaver skin. With Franz cells, in each Franz diffusion cell a disc (diameter of 25 mm) of human cadaver skin is placed on the receptor compartment. A transdermal delivery system is cut the same size as the skin and placed over the diffusion area in the center of the receptor. The donor compartment is then added and clamped to the assembly. At time 0, receptor medium solution 14 mL is added into the receptor compartment and the cell maintained at 32° C. Samples of the receptor compartment are taken periodically to determine the skin flux and analyzed by HPLC.

The pharmacokinetics studies of a transdermal patch containing galantamine, an acetylcholinesterase inhibitor were assessed in 12 healthy volunteers. The transdermal system is apply to the healthy subjects topically on their arm or lower back, or upper back for a day. The blood samples were collected periodically. Measurements of galantamine serum levels were performed and analyzed LC-MS.

### 6.1.1 Human Pharmacokinetics Profile

Human Pharmacokinetics profiles were determined for various formulations tested with the formulations provided in Table 1.

TABLE 1

| | AUC0-t | |
|---|---|---|
| Formulation No. | (h · ng/mL) | Cmax (ng/mL) |
| 004-083-001 | 305.45 ± 133.74 | 13.00 ± 5.15 |
| 004-083-003 | 403.52 ± 191.77 | 16.86 ± 7.01 |
| 048-018-001 | 65.73 ± 71.67 | 2.79 ± 3.09 |
| 048-025-001 | 84.87 ± 85.28 | 3.69 ± 3.03 |
| 048-019-001 | 142.41 ± 118.37 | 6.52 ± 5.33 |
| 048-026-001 | 177.93 ± 182.84 | 8.52 ± 8.26 |

### 6.1.2. Adhesive Formulations

Various adhesives were tested to determine the maximum amount of galantamine could be dissolved. The results are provided below in Table 2.

TABLE 2

| | | Contain | | | | |
|---|---|---|---|---|---|---|
| | | Vinyl | Functional | | Cross | API |
| Adhesive | Description | Acetate | Group | Curing | Linker | Loading |
| Duro Tak | acrylic | N | None | N | N | <10% |
| 87-9301 | | | | | | |
| Duro Tak | acrylate- | Y | None | N | N | <10% |
| 487-098 | vinylacetate | | | | | |
| Duro Tak | acrylic | N | —OH | N | N | <10% |
| 87-2510 | | | | | | |
| Duro Tak | acrylate- | Y | —OH | Y | Y | <10% |
| 87-2516 | vinylacetate | | | | | |
| Duro Tak | acrylate- | Y | —OH | N | N | <10% |
| 87-4287 | vinylacetate | | | | | |
| Duro Tak | acrylic | N | —COOH | | N | <10% |
| 87-235A | | | | | | |
| Duro Tak | acrylic | N | —COOH | Y | Y | <10% |
| 87-2852 | | | | | | |
| Duro Tak | acrylic | N | —COOH | N | N | <10% |
| 87-2353 | | | | | | |
| Duro Tak | acrylate- | Y | —COOH | Y | Y | <20% |
| 387-2054 | vinylacetate | | | | | |
| Duro Tak | acrylate- | Y | —COOH | Y | Y | 22% |
| 87-2196 | vinylacetate | | | | | |
| Duro Tak | acrylate- | Y | —COOH/—OH | Y | Y | <8% |
| 87-2979 | vinylacetate | | | | | |
| Duro Tak | acrylic-rubber | N | | N | N | <5% |
| 87-502b | hybrid | | | | | |
| Duro Tak | acrylic-rubber | N | | Y | N | <5% |
| 87-504b | hybrid | | | | | |
| Duro Tak | Polyisobutylene | | | | | <3% |
| 87-6908 | (PIB) rubber | | | | | |
| | composition | | | | | |
| Duro Tak | Styrene-butadiene | | | | | <3% |
| 87-6911 | block copolymer | | | | | |
| | rubber and | | | | | |
| | hydrogenated | | | | | |
| | synthetic | | | | | |
| | hydrocarbon | | | | | |
| | tackifier | | | | | |
| BIO-PSA | Amine-Compatible | | | | | 3% |
| 4102 | Silicon Adhesive | | | | | |
| BIO-PSA | Amine-Compatible | | | | | |
| 4202 | Silicon Adhesive | | | | | 3% |
| BIO-PSA | Amine-Compatible | | | | | |
| 4302 | Silicon Adhesive | | | | | 3% |
| BIO-PSA | Silicon Adhesive | | | | | 3% |
| 4602 | | | | | | |

### 6.1.3. Enhancer Compositions

Various enhancers were tested to determine the maximum amount of galantamine could be dissolved. The results are provided in Table 3.

TABLE 3

| Enhancer | Category | Solubility (mg/mL) |
|---|---|---|
| Squalane | solvent | 1.3 |
| Oleyl Oleate | Fatty acid ester | 5.2 |
| IPP | Fatty acid ester | 6.0 |
| IPM | Fatty acid ester | 7.6 |
| Span 20 | classical surfactants | 7.8 |
| Limonene | solvent | 9.3 |
| Ethyl Oleate | fatty acid ester | 9.6 |
| Methyl Laurate | Fatty acid ester | 12.6 |
| Labrafac PG | Fatty acid ester | 15.7 |
| Labrafac Lipophile WL1349 | Fatty acid | 16.7 |
| Octanol | Fatty alcohol | 24.1 |
| Lauryl Alcohol | Fatty alcohol | 30.3 |
| Brij 30 | classical surfactants | 34.8 |
| Oleyl Alcohol | Fatty alcohol | 40.1 |
| Lauroglycol FCC | Fatty acid ester | 40.6 |
| Tween 80 | classical surfactants | 41.3 |
| Propylene Glycol | solvent | 112.9 |
| Transcutal P | solvent | 122.4 |
| Oleic Acid | Fatty acid | 139.2 |

### 6.1.4. Antioxidant Effect

Various of antioxidant were tested under normal condition, and stress conditions to determine the antioxidant effect to stabilize the galantamine. The results are provided in Table 4.

| | Total Impurity Amount % | | | |
|---|---|---|---|---|
| Antioxidant | 25′C | 60′C-14 D | 80′C-1 D | 80′C-4 D |
| Control (without antioxidant) | 0.11 | 1.20 | 0.63 | 1.84 |
| Vitamin E | 0.12 | 0.84 | 0.69 | 1.77 |
| Ascorbyl Palmitate | 0.12 | 0.58 | 0.28 | 1.13 |
| Bronopol | 0.18 | 0.81 | 0.69 | 1.82 |
| Butylated Hydroxytoluene | 0.11 | 0.33 | 0.51 | 1.18 |
| (BHT) | | | | |
| Erythorbic Acid | 0.50 | 1.35 | 0.74 | 0.87 |
| Monothioglycerol | 0.16 | 1.09 | 0.51 | 1.49 |
| Sodium metabisulfite | 0.17 | 0.45 | 0.14 | 0.93 |
| Sodium Sulfite | 0.14 | 1.04 | 0.74 | 1.69 |
| Sulfur Dioxide | 0.16 | 1.04 | 0.54 | 1.83 |
| Sodium Thiosulfate | 0.13 | 1.02 | 0.67 | 2.20 |

### 6.1.5. Skin Permeation

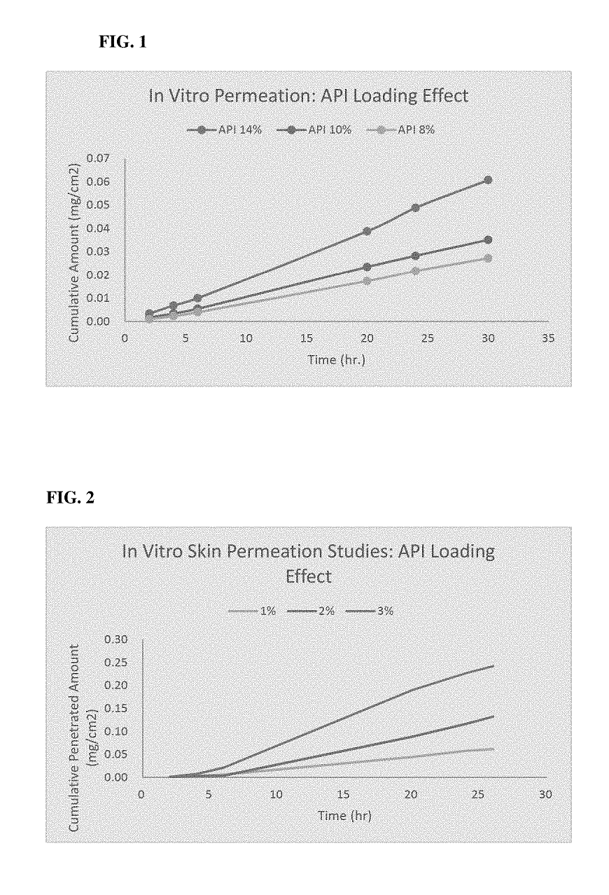

Skin permeation of various formulations were tested. The results are provided in FIGS. 1, and 2 and Tables 5 and 6. Higher the amount of the galantamine in the adhesives will increase the penetration, but when over the saturation point, the transdermal system will crystalize, and result a low penetration rate.

When adding an enhancer, it might improve the penetration rate. An enhancer improves the penetration rate, but functional enhancer might not have the same effect. When increase the amount of the enhancer, it might not improve the penetration rate. When combine with any two enhancers, the penetration rate does not always improve. As the results are indicated in Table 5 and 6.

TABLE 5-1
One Enhancer Effect with Galantamine at 14%
W/W in Acrylate-Vinyl Acetate Copolymer

| | Enhancer | | Enhancement |
|---|---|---|---|
| Enhancer | % w/w | Category | Ratio |
| Control | 0 | | — |
| Labrafil M 1944CS | 3 | Fatty acid | 1 |
| IPM | 5 | Fatty acid ester | 1 |
| Lauroglycol FCC | 5 | Fatty acid ester | 1 |
| Kolliphor RH40 | 1 | classical surfactants | 1 |
| Oleyl Oleate | 5 | Fatty acid ester | 2 |
| Labrafac Lipophile | 5 | Fatty acid | 3 |
| WL1349 | | | |
| Labrafac PG | 5 | Fatty acid ester | 3 |
| Transcutol P | 5 | solvent | 4 |
| Propylene Glycol | 5 | solvent | 4 |
| Kolliphor RH 40 | 10 | classical surfactants | 5 |
| Lauroglycol FCC | 10 | Fatty acid ester | 5 |
| Labrafac Lipophile | 20 | Fatty acid | 6 |
| WL1349 | | | |
| Propylene Glycol | 10 | solvent | 7 |
| Labrafac Lipophile | 10 | Fatty acid | 9 |
| WL1349 | | | |

TABLE 5-2
One Enhancer Effect with Galantamine at 3%
W/W in Styrene-butadiene block copolymer rubber

| | | | Enhancement |
|---|---|---|---|
| Enhancer | Category | Enhancer % w/w | Ratio |
| Control | | | |
| Brij 30 | Surfactants | 3 | 2 |
| Ethyl Oleate | Fatty acid ester | 3 | 2 |
| IPM | Fatty acid ester | 3 | 2 |
| IPP | Fatty acid ester | 3 | 2 |
| Lauroglycol | Fatty acid ester | 3 | 2 |
| Oleic Acid | Fatty acid | 3 | 1 |
| Oleyl Oleate | Fatty acid ester | 3 | 2 |
| Squalane | Surfactants | 3 | 1 |
| Triacetin | Solvent | 3 | 0 |
| Vitamin E TPGS | Surfactants | 3 | 1 |

TABLE 6-1
Two Enhancer Effect with Antioxidant with Galantamine at
14% W/W in Acrylate-Vinyl Acetate Copolymer

| | Enhancer 1 | | Enhancer 2 | | Enhancement |
|---|---|---|---|---|---|
| Enhancer 1 | % w/w | Enhancer 2 | % w/w | Antioxidant | Ratio |
| Control | | | | | — |
| Propylene | 5 | Lauroglycol FCC | 5 | Butylated | 3 |
| Glycol | | | | Hydroxytoluene | |
| Propylene | 5 | Oleyl Oleate | 5 | Butylated | 10 |
| Glycol | | | | Hydroxytoluene | |
| Propylene | 5 | Labrafac PG | 5 | Butylated | 7 |
| Glycol | | | | Hydroxytoluene | |
| Propylene | 5 | Labrafac | 5 | Butylated | 5 |
| Glycol | | Lipophile | | Hydroxytoluene | |
| | | WL1349 | | | |
| Transcutol P | 5 | Lauroglycol FCC | 5 | Butylated | 4 |
| | | | | Hydroxytoluene | |

TABLET 6-2
Two Enhancer Effect with Antioxidant with Galantamine at 2.5%
W/W in Styrene-butadiene block copolymer rubber

| | Enhancer 1 | | Enhancer 2 | Enhancement |
|---|---|---|---|---|
| Enhancer1 | % w/w | Enhancer 2 | % w/w | Ratio |
| Control | | | | |
| Labrafac | 5 | Oleyl Oleate | 5 | 2 |
| Lipophile | | | | |
| WL1349 | | | | |
| Labrafac PG | 5 | Oleyl Oleate | 5 | 2 |
| Ethyl Oleate | 5 | Oleyl Oleate | 5 | 2 |
| Lauroglycol | 5 | Oleyl Oleate | 5 | 2 |
| Brij30 | 5 | Oley Oleate | 5 | 2 |
| Labrafac PG | 8 | Oleyl Oleate | 3 | 2 |
| Ethyl Oleate | 8 | Oleyl Oleate | 3 | 2 |
| Lauroglycol | 8 | Oleyl Oleate | 3 | 1 |
| Lauroglycol | 5 | Oleyl Oleate | 5 | 1 |
| Ethyl Oleate | 3 | Oleyl Oleate | 3 | 1 |
| Lauroglycol | 3 | Oleyl Oleate | 3 | 1 |
| Ethyl Oleate | 3 | Lauroglycol | 3 | 1 |
| Ethyl Oleate | 3 | Oleyl Oleate | 3 | 6 |
| Lauroglycol | 3 | Oleyl Oleate | 3 | 3 |
| Ethyl Oleate | 3 | Lauroglycol | 3 | 1 |
| Lauroglycol | 4 | Oleyl Oleate | 4 | 3 |
| Lauroglycol | 4 | Oleyl Oleate | 3 | 2 |
| Lauroglycol | 5 | Oleyl Oleate | 10 | 3 |

A co-solvent helps dissolving the drug in the adhesive but it might also help with the penetration rate. As in Table 7, Transcutol® P helps to dissolve galantamine in the adhesive and also improves the penetration rate.

TABLE 7
Co-Solvent Effect in Styrene-butadiene block copolymer
rubber Adhesive, Galantamine at 2.5% w/w

| % Enhancer | Antioxidant | Co-solvent 5% | Enhancement Ratio |
|---|---|---|---|
| 5% Ethyl Oleate | BHT | | — |
| 5% Ethyl Oleate | BHT | Transcutol P | 5 |

### 6.1.6. Crystallization

Various adhesives were tested to find the optimal drug solubility to optimize the penetration. Polymers of same functional group, does not demonstrate same observation. To prevent the crystallization problem, enhancers act as solubilizer to increase the amount of the galantamine to be dissolved in the adhesive. The results are shown in Tables 8 and 9.

TABLE 8
Without Enhancer

| Adhesive | Galantamine w/w | Observation |
|---|---|---|
| Duro-Tak 387-2054 | 10 | no crystallization |
| Duro-Tak 387-2054 | 22 | crystallization |
| Duro-Tak 387-2196 | 10 | no crystallization |
| Duro-Tak 387-2196 | 22 | crystallization |
| Duro-Tak 502b | 5 | crystallization |
| Duro-Tak 504b | 10 | crystallization |
| Duro-Tak 87-6911 | 1 | no crystallization |
| Duro-Tak 87-6911 | 9 | crystallization |
| Duro-Tak 87-6911 | 20 | segregation |

TABLE 9
With Enhancer

| | Galantamine | Oleic Acid | Oleyl Oletae | Triacetin | |
|---|---|---|---|---|---|
| Adhesive | % w/w | % w/w | % w/w | % w/w | Observation |
| Duro Tak 87-9301 | 7 | | 10 | | crystallization |
| Duro Tak 87-9301 | 7 | 5 | 5 | | crystallization |
| Duro Tak 87-9301 | 7 | 10 | 5 | | no crystallization |
| Duro Tak 87-9301 | 8 | | 10 | | crystallization |
| Duro Tak 87-9301 | 8 | 5 | 5 | | crystallization |
| Duro Tak 87-9301 | 8 | 10 | | | no crystallization |
| Duro Tak 87-9301 | 8 | | 5 | | crystallization |
| Duro Tak 87-9301 | 8 | | 5 | 5 | crystallization |
| Duro Tak 87-9301 | 8 | 10 | 5 | | no crystallization |
| Duro Tak 87-9301 | 10 | | 5 | | crystallization |

Exemplary Systems and Methods are Set Out in the Following Items:

Item 1. A transdermal delivery system which comprises a drug-containing matrix layer comprising:
(i) galantamine or its pharmaceutically acceptable salt;
(ii) an adhesive comprising an acrylic polymer without a functional group or an acrylate-vinyl acetate polymer having at least one carboxyl functional group (COOH);
(iii) an enhancer composition including: (a) oleyl oleate and oleic acid; (b) oleyl oleate and propylene glycol and an antioxidant; or (c) a medium chain fatty acid triglyceride and an antioxidant.
Item 2. The transdermal delivery system of item 1, wherein the medium chain fatty acid triglyceride comprises about 50-80% of caprylic acid and about 20-50% of capric acid.
Item 3. The transdermal delivery system of any one of the preceding items, wherein the transdermal delivery system consists of a backing layer, the drug-containing matrix layer, and a release layer.
Item 4. The transdermal delivery system of any one of the preceding items, wherein the galantamine or its pharmaceutically acceptable salt is about 7 to about 14% by weight based on the total weight of the drug-containing matrix layer.
Item 5. The transdermal delivery system of any one of the preceding items, wherein the galantamine or its pharmaceutically acceptable salt is about 7, about 8 or about 14% by weight based on the total weight of the drug-containing matrix layer.
Item 6. The transdermal delivery system of any one of the preceding items, wherein the adhesive is about 75-78% by weight based on the total weight of the drug-containing matrix layer.
Item 7. The transdermal delivery system of any one of the preceding items, wherein the acrylic polymer has a glass transition temperature from about −15° C. to about −30° C.
Item 8. The transdermal delivery system of any one of the preceding items, wherein the acrylate-vinylacetate polymer has a glass transition temperature from about −30° C. to about −60° C.
Item 9. The transdermal delivery system of any one of the preceding items, wherein the oleic acid is about 10% by weight based on the total weight of the drug-containing matrix layer.
Item 10. The transdermal delivery system of any one of the preceding items, wherein the oleyl oleate is about 5% by weight based on the total weight of the drug-containing matrix layer.
Item 11. The transdermal delivery system of any one of the preceding items, wherein the medium chain fatty acid triglyceride is about 10% by weight based on the total weight of the drug-containing matrix layer.
Item 12. The transdermal delivery system of any one of the preceding items, wherein the antioxidant is butylated hydoxytoluene.
Item 13. The transdermal delivery system of any one of the preceding items, wherein the antioxidant is about 0.05% by weight based on the total weight of the drug-containing matrix layer.
Item 14. The transdermal delivery system of any one of the preceding items, wherein the drug-containing matrix layer has a thickness from about 20 μm to about 48 μm.
Item 15. The transdermal delivery system of any one of the preceding items, wherein the system has a flux of about 8-10 μg/cm² hr.
Item 16. The transdermal delivery system of any one of the preceding items, wherein the galantamine or its pharmaceutically acceptable salt is about 7-8% by weight, wherein the adhesive is about 78% by weight, wherein the oleic acid is about 10% by weight, wherein the oleyl oleate is about 5% by weight, based on the total weight of the drug-containing matrix layer, wherein the drug-containing matrix layer has a thickness from about 20-48 μm.
Item 17. A transdermal delivery system which comprises a drug-containing matrix layer comprising:
(i) about 7-8% of galantamine or its pharmaceutically acceptable salt;
(ii) an adhesive comprising an acrylic polymer without a functional group:
(iii) an enhancer composition comprising: (a) about 10% oleic acid and about 5% oleyl oleate, wherein the transdermal system has a thickness of about 20-48 μm.
Item 18. The transdermal delivery system of any one of the preceding items, wherein the adhesive has a glass transition temperature from about −15° C. to about −30° C.
Item 19. A transdermal delivery system which comprises a drug-containing matrix layer comprising:
(i) about 14% of galantamine or its pharmaceutically acceptable salt;
(ii) an adhesive comprising an acrylate-vinlyacetate polymer having at least one functional COOH group;
(iii) an enhancer composition comprising about 10% medium chain fatty acid triglyceride;
(iv) about 0.05% antioxidant,
wherein the transdermal system has a thickness of about 15-45 μm.
Item 20. The transdermal delivery system of any one of the preceding items wherein the adhesive has a glass transition temperature from about −30° C. to about −60° C.
Item 21. The transdermal delivery system of any one of the preceding items, wherein the antioxidant is butylated hydroxytoluene, vitamin E, ascorbyl palmitate, bronopol, erythorbic acid, monothioglycerol, sodium metabisulfite, sodium sulfite, sulfur dioxide or sodium thiosulfate.
Item 22. The transdermal delivery system of any one of the preceding items, wherein the system has a flux of about 8 μg/cm² hr.
Item 23. A transdermal delivery system which comprises a drug-containing matrix layer comprising:
(i) about 14% of galantamine or its pharmaceutically acceptable salt;
(ii) an adhesive comprising an acrylate-vinlyacetate polymer having at least one functional COOH group;
(iii) an enhancer composition comprising about 5% polypylene glycol and about 5% oleyl oleate;
(iv) about 0.05% antioxidant,
wherein the transdermal system has a thickness of about 15-45 μm.
Item 24. The transdermal delivery system of any one of the preceding items, wherein the adhesive has a glass transition temperature from about −30° C. to about −60° C.
Item 25. The transdermal delivery system of any one of the preceding items, wherein the system has a flux of about 9.3 to about 9.5 μg/cm² hr.
Item 26. The transdermal delivery system of any one of the preceding items, wherein the galantamine or its pharmaceutically acceptable salt is about 14% by weight, wherein the adhesive is about 76% by weight, wherein the oleyl oleate is about 5% by weight, wherein the medium chain fatty acid triglyceride is about 10%, wherein the polyethylene glycol is about 5%, wherein the antioxidant is butylated hydroxytoluene that is about 0.05% by weight, based on the total weight of the drug-containing matrix layer, and wherein the drug-containing matrix layer has a thickness of about 15-45 μm.
Item 27. The transdermal delivery system of any one of the preceding items wherein the system has a flux of about 1-15 μg/cm² hr.
Item 28. A transdermal delivery system which comprises a drug-containing matrix layer comprising:
(i) galantamine or its pharmaceutically acceptable salt;
(ii) an adhesive comprising styrene butadiene-styrene block copolymer;
(iii) an enhancer composition comprising oleyl oleate and propyleneglycol monolaurate; and
(iv) an antioxidant, a solvent and an adhesive modifier.
Item 29. The transdermal delivery system of any one of the preceding items, wherein the antioxidant is butylated hydroxytoluene, vitamin E, ascorbyl palmitate, bronopol, erythorbic acid, monothioglycerol, sodium metabisulfite, sodium sulfite, sulfur dioxide, sodium thiosulfate and combinations thereof, wherein the solvent is diethylene glycol monoethyl ether, and wherein the adhesive modifier is a cyclic terpene.
Item 30. The transdermal delivery system of any one of the preceding items, wherein the transdermal delivery system consists of a backing layer, the drug-containing matrix layer, and a release layer.
Item 31. The transdermal delivery system of any one of the preceding items, wherein the galantamine or its pharmaceutically acceptable salt is about 0.01 to about 5% by weight based on the total weight of the drug-containing matrix layer.
Item 32. The transdermal delivery system of any one of the preceding items, wherein the styrene-butadiene-styrene block copolymer is about 60-97.5% by weight based on the total weight of the drug-containing matrix layer.
Item 33. The transdermal delivery system of any one of the preceding items, wherein the enhancer composition is about 3-15% by weight based on the total weight of the drug-containing matrix layer.
Item 34. The transdermal delivery system of any one of the preceding items, wherein the propyleneglycol monolaurate is about 0.1-15% by weight based on the total weight of the drug-containing matrix layer.
Item 35. The transdermal delivery system of any one of the preceding items, wherein the oleyl oleate is about 0.1-20% by weight based on the total weight of the drug-containing matrix layer.
Item 36. The transdermal delivery system of any one of the preceding items, wherein the antioxidant is butylated hydroxytoluene and is about 0.001-0.5% by weight based on the total weight of the drug-containing matrix layer.
Item 37. The transdermal delivery system of any one of the preceding items, wherein the solvent is diethylene glycol monoethyl ether and is about 0.1-20% by weight based on the total weight of the drug-containing matrix layer.
Item 38. The transdermal delivery system of any one of the preceding items, wherein the adhesive modifier is a cyclic terpene and is about 0.1-15% by weight based on the total weight of the drug-containing matrix layer.
Item 39. The transdermal delivery system of any one of the preceding items, wherein the propyleneglycol monolaurate is propyleneglycol monolaurate type I or propyleneglycol monolaurate type II.
Item 40. The transdermal delivery system of any one of the preceding items, wherein the drug-containing matrix layer has a thickness from about 45 μm to about 85 μm.
Item 41. The transdermal delivery system of any one of the preceding items, wherein the galantamine or its pharmaceutically acceptable salt is about 2.5% by weight, wherein the styrene-butadiene-styrene block copolymer is about 60-97.5% by weight, wherein the propyleneglycol monolaurate is about 0.1-15% by weight, wherein oleyl oleate is about 0.1-20% by weight, wherein the antioxidant is butylated hydroxyl toluene and is about 0.001-0.5% by weight, wherein the solvent is diethylene glycol monoethyl ether and is about 0.1-20% by weight, and wherein the adhesive modifier is cyclic terpene and is about 0.1-15% by weight, based on the total weight of the drug-containing matrix layer.
Item 42. The transdermal delivery system of any one of the preceding items, wherein the propyleneglycol monolaurate is propyleneglycol monolaurate type I or propyleneglycol monolaurate type II.
Item 43. A transdermal delivery system which comprises a drug-containing matrix layer comprising:
(i) about 0.01-5% of galantamine or its pharmaceutically acceptable salt;
(ii) about 60-97.7% of an adhesive comprising styrene butadiene-styrene block copolymer;
(iii) an enhancer composition comprising about 0.1-20% oleyl oleate and about 0.1-15% propyleneglycol monolaurate;
(v) about 0.001-0.5% of an antioxidant;
(vi) about 0.1-20% solvent; and
(vii) about 0.1-15% adhesive modifier.
Item 44. The transdermal delivery system of any one of the preceding items, wherein the galantamine is about 2.5% by weight based on the total weight of the drug-containing matrix layer.
Item 45. The transdermal delivery system of any one of the preceding items, wherein the enhancer composition is about 3-8% by weight based on the total weight of the drug-containing matrix layer.
Item 46. The transdermal delivery system of any one of the preceding items, wherein the propyleneglycol monolaurate is propyleneglycol monolaurate type I or propyleneglycol monolaurate type II.
Item 47. The transdermal delivery system of any one of the preceding items, wherein the antioxidant is butylated hydroxyl toluene.
Item 48. The transdermal delivery system of any one of the preceding items, wherein the solvent is diethylene glycol monoethyl ether.
Item 49. The transdermal delivery system of any one of the preceding items, wherein the adhesive modifier is cyclic terpene.
Item 50. The transdermal delivery system of any one of the preceding items, wherein the enhancer composition is about 3, about 5 or about 8% by weight based on the total weight of the drug-containing matrix layer.

The disclosure is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A transdermal delivery system which comprises a drug-containing matrix layer comprising:
(i) galantamine or its pharmaceutically acceptable salt;
(ii) an adhesive comprising an acrylic polymer without a functional group or an acrylate-vinyl acetate polymer having at least one carboxyl functional group (COOH);
(iii) an enhancer composition including: (a) oleyl oleate and oleic acid; (b) oleyl oleate and propylene glycol and an antioxidant; or (c) a medium chain fatty acid triglyceride and an antioxidant.

2. The transdermal delivery system of claim 1, wherein the medium chain fatty acid triglyceride comprises about 50-80% of caprylic acid and about 20-50% of capric acid.

3. The transdermal delivery system of claim 1, wherein the transdermal delivery system consists of a backing layer, the drug-containing matrix layer, and a release layer.

4. The transdermal delivery system of claim 1, wherein the galantamine or its pharmaceutically acceptable salt is about 7 to about 14% by weight based on the total weight of the drug-containing matrix layer.

5. The transdermal delivery system of claim 4, wherein the galantamine or its pharmaceutically acceptable salt is about 7, about 8 or about 14% by weight based on the total weight of the drug-containing matrix layer.

6. The transdermal delivery system of claim 1, wherein the adhesive is about 75-78% by weight based on the total weight of the drug-containing matrix layer.

7. The transdermal delivery system of claim 1, wherein the acrylic polymer has a glass transition temperature from about −15° C. to about −30° C.

8. The transdermal delivery system of claim 1, wherein the acrylate-vinylacetate polymer has a glass transition temperature from about −30° C. to about −60° C.

9. The transdermal delivery system of claim 1, wherein the oleic acid is about 10% by weight based on the total weight of the drug-containing matrix layer.

10. The transdermal delivery system of claim 1, wherein the oleyl oleate is about 5% by weight based on the total weight of the drug-containing matrix layer.

11. The transdermal delivery system of claim 1, wherein the medium chain fatty acid triglyceride is about 10% by weight based on the total weight of the drug-containing matrix layer.

12. The transdermal delivery system of claim 1, wherein the antioxidant is butylated hydoxytoluene.

13. The transdermal delivery system of claim 1, wherein the antioxidant is about 0.05% by weight based on the total weight of the drug-containing matrix layer.

14. The transdermal delivery system of claim 1, wherein the drug-containing matrix layer has a thickness from about 20 μm to about 48 μm.

15. The transdermal delivery system of claim 1, wherein the system has a flux of about 8-10 μg/cm2 hr.

16. The transdermal delivery system of claim 1, wherein the galantamine or its pharmaceutically acceptable salt is about 7-8% by weight, wherein the adhesive is about 78% by weight, wherein the oleic acid is about 10% by weight, wherein the oleyl oleate is about 5% by weight, based on the total weight of the drug-containing matrix layer, wherein the drug-containing matrix layer has a thickness from about 20-48 μm.

17. A transdermal delivery system which comprises a drug-containing matrix layer comprising:
(i) about 7-8% of galantamine or its pharmaceutically acceptable salt;
(ii) an adhesive comprising an acrylic polymer without a functional group;
(iii) an enhancer composition comprising: (a) about 10% oleic acid and about 5% oleyl oleate,
wherein the transdermal system has a thickness of about 20-48 μm.

18. The transdermal delivery system of claim 17, wherein the adhesive has a glass transition temperature from about −15° C. to about −30° C.

19. A transdermal delivery system which comprises a drug-containing matrix layer comprising:
(i) about 14% of galantamine or its pharmaceutically acceptable salt;
(ii) an adhesive comprising an acrylate-vinlyacetate polymer having at least one functional COOH group;
(iii) an enhancer composition comprising about 10% medium chain fatty acid triglyceride;
(iv) about 0.05% antioxidant,
wherein the transdermal system has a thickness of about 15-45 μm.

20. The transdermal delivery system of claim 19 wherein the adhesive has a glass transition temperature from about −30° C. to about −60° C.

21. The transdermal delivery system of claim 19, wherein the antioxidant is butylated hydroxytoluene, vitamin E, ascorbyl palmitate, bronopol, erythorbic acid, monothioglycerol, sodium metabisulfite, sodium sulfite, sulfur dioxide or sodium thiosulfate.

22. The transdermal delivery system of claim 19, wherein the system has a flux of about 8 μg/cm2 hr.

23. A transdermal delivery system which comprises a drug-containing matrix layer comprising:
(i) about 14% of galantamine or its pharmaceutically acceptable salt;
(ii) an adhesive comprising an acrylate-vinlyacetate polymer having at least one functional COOH group;
(iii) an enhancer composition comprising about 5% polypylene glycol and about 5% oleyl oleate;
(iv) about 0.05% antioxidant,
wherein the transdermal system has a thickness of about 15-45 μm.

24. The transdermal delivery system of claim 23, wherein the adhesive has a glass transition temperature from about −30° C. to about −60° C.

25. The transdermal delivery system of claim 23, wherein the system has a flux of about 9.3 to about 9.5 μg/cm2 hr.

26. The transdermal delivery system of claim 1, wherein the galantamine or its pharmaceutically acceptable salt is about 14% by weight, wherein the adhesive is about 76% by weight, wherein the oleyl oleate is about 5% by weight, wherein the medium chain fatty acid triglyceride is about 10%, wherein the polyethylene glycol is about 5%, wherein the antioxidant is butylated hydroxytoluene that is about 0.05% by weight, based on the total weight of the drug-containing matrix layer, and wherein the drug-containing matrix layer has a thickness of about 15-45 μm.

27. The transdermal delivery system of claim 26 wherein the system has a flux of about 1-15 μg/cm2 hr.

28. A transdermal delivery system which comprises a drug-containing matrix layer comprising:
(i) galantamine or its pharmaceutically acceptable salt;
(ii) an adhesive comprising styrene butadiene-styrene block copolymer;
(iii) an enhancer composition comprising oleyl oleate and propyleneglycol monolaurate; and
(iv) an antioxidant, a solvent and an adhesive modifier.

29. The transdermal delivery system of claim 28, wherein the antioxidant is butylated hydroxytoluene, vitamin E, ascorbyl palmitate, bronopol, erythorbic acid, monothioglycerol, sodium metabisulfite, sodium sulfite, sulfur dioxide, sodium thiosulfate and combinations thereof, wherein the solvent is diethylene glycol monoethyl ether, and wherein the adhesive modifier is a cyclic terpene.

30. The transdermal delivery system of claim 28, wherein the transdermal delivery system consists of a backing layer, the drug-containing matrix layer, and a release layer.

31. The transdermal delivery system of claim 28, wherein the galantamine or its pharmaceutically acceptable salt is about 0.01 to about 5% by weight based on the total weight of the drug-containing matrix layer.

32. The transdermal delivery system of claim 28, wherein the styrene-butadiene-styrene block copolymer is about 60-97.5% by weight based on the total weight of the drug-containing matrix layer.

33. The transdermal delivery system of claim 28, wherein the enhancer composition is about 3-15% by weight based on the total weight of the drug-containing matrix layer.

34. The transdermal delivery system of claim 28, wherein the propyleneglycol monolaurate is about 0.1-15% by weight based on the total weight of the drug-containing matrix layer.

35. The transdermal delivery system of claim 28, wherein the oleyl oleate is about 0.1-20% by weight based on the total weight of the drug-containing matrix layer.

36. The transdermal delivery system of claim 28, wherein the antioxidant is butylated hydroxytoluene and is about 0.001-0.5% by weight based on the total weight of the drug-containing matrix layer.

37. The transdermal delivery system of claim 28, wherein the solvent is diethylene glycol monoethyl ether and is about 0.1-20% by weight based on the total weight of the drug-containing matrix layer.

38. The transdermal delivery system of claim 28, wherein the adhesive modifier is a cyclic terpene and is about 0.1-15% by weight based on the total weight of the drug-containing matrix layer.

39. The transdermal delivery system of claim 28, wherein the propyleneglycol monolaurate is propyleneglycol monolaurate type I or propyleneglycol monolaurate type II.

40. The transdermal delivery system of claim 28, wherein the drug-containing matrix layer has a thickness from about 45 μm to about 85 μm.

41. The transdermal delivery system of claim 28, wherein the galantamine or its pharmaceutically acceptable salt is about 2.5% by weight, wherein the styrene-butadiene-styrene block copolymer is about 60-97.5% by weight, wherein the propyleneglycol monolaurate is about 0.1-15% by weight, wherein oleyl oleate is about 0.1-20% by weight, wherein the antioxidant is butylated hydroxyl toluene and is about 0.001-0.5% by weight, wherein the solvent is diethylene glycol monoethyl ether and is about 0.1-20% by weight, and wherein the adhesive modifier is cyclic terpene and is about 0.1-15% by weight, based on the total weight of the drug-containing matrix layer.

42. The transdermal delivery system of claim 41, wherein the propyleneglycol monolaurate is propyleneglycol monolaurate type I or propyleneglycol monolaurate type II.

43. A transdermal delivery system which comprises a drug-containing matrix layer comprising:
(i) about 0.01-5% of galantamine or its pharmaceutically acceptable salt;
(ii) about 60-97.7% of an adhesive comprising styrene butadiene-styrene block copolymer,
(iii) an enhancer composition comprising about 0.1-20% oleyl oleate and about 0.1-15% propyleneglycol monolaurate;
(v) about 0.001-0.5% of an antioxidant;
(vi) about 0.1-20% solvent; and
(vii) about 0.1-15% adhesive modifier.

44. The transdermal delivery system of claim 43, wherein the galantamine is about 2.5% by weight based on the total weight of the drug-containing matrix layer.

45. The transdermal delivery system of claim 43, wherein the enhancer composition is about 3-8% by weight based on the total weight of the drug-containing matrix layer.

46. The transdermal delivery system of claim 43, wherein the propyleneglycol monolaurate is propyleneglycol monolaurate type I or propyleneglycol monolaurate type II.

47. The transdermal delivery system of claim 43, wherein the antioxidant is butylated hydroxyl toluene.

48. The transdermal delivery system of claim 43, wherein the solvent is diethylene glycol monoethyl ether.

49. The transdermal delivery system of claim 43, wherein the adhesive modifier is cyclic terpene.

50. The transdermal delivery system of claim 45, wherein the enhancer composition is about 3, about 5 or about 8% by weight based on the total weight of the drug-containing matrix layer.
